(19) United States
(12) Patent Application Publication
(10) Pub. No.: US 2024/0409614 A1
(43) Pub. Date: Dec. 12, 2024

(54) VARIANTS OF ALPHA-1-ANTITRYPSIN

(71) Applicant: DANMARKS TEKNISKE UNIVERSITET, Kongens Lyngby (DK)

(72) Inventors: Sanne Schoffelen, Kongens Lyngby (DK); Bjørn Gunnar Voldborg, Kongens Lyngby (DK); Gerry McElvaney, Kongens Lyngby (DK)

(21) Appl. No.: 18/700,638

(22) PCT Filed: Nov. 9, 2022
(86) PCT No.: PCT/EP2022/081255

§ 371 Date: Apr. 11, 2024

(30) Foreign Application Priority Data
Nov. 9, 2021 (EP) 21207135.1

(51) Int. Cl. C07K 14/81; A61K 38/00

(52) U.S. Cl. CPC C07K 14/8125; A61K 38/00; C07K 2319/91

(57) ABSTRACT

Provided are variants of alpha-1-antitrypsin comprising mutations which render the variants oxidation-as well as protease-resistant, polynucleotides encoding said variants, methods of producing the variants and the variants for use in the treatment of alpha-1-antitrypsin deficiency, cystic fibrosis and chronic obstructive pulmonary disease.

### CROSS-REFERENCE TO RELATED APPLICATIONS

This application is a U.S. national phase application pursuant to 35 U.S.C. § 371 of International Patent Application No. PCT/EP2022/081255, filed Nov. 9, 2022, which claims priority to European Patent Application No. 21207135.1, filed Nov. 9, 2021, the disclosures of which are incorporated herein by reference in its entirety.

### INCORPORATION BY REFERENCE OF MATERIAL SUBMITTED ELECTRONICALLY

The application is submitted with a sequence listing in electronic format. The sequence listing is provided as a file entitled “70116_SeqListing.xml,” created Nov. 7, 2022, which is 33,437 bytes in size. The information in the sequence listing is incorporated herein by reference in its entirety.

### FIELD OF THE INVENTION

The present invention relates to variants of human alpha-1-antitrypsin and their use in the treatment of alpha-1-antitrypsin deficiency and other diseases. The variants may comprise human N-glycans and have an increased resistance against oxidation and proteolysis.

### BACKGROUND OF THE INVENTION

Alpha-1-antitrypsin (A1AT) belongs to the family of serine protease inhibitors (serpins). It is a 52 kDa single-chain glycoprotein that is produced in and secreted by the liver. Its main function is to inhibit neutrophil elastase in the lungs. Insufficient inhibition of elastase leads to lung damage. A1AT is currently used as a biopharmaceutical to treat A1AT deficiency, particularly A1AT purified from fractionated human plasma.

It has previously been described that A1AT may advantageously be expressed in glyco-engineered Chinese hamster ovary (CHO) cell lines by which the protein gets a humanized N-glycosylation profile (Amann et al., Metab. Eng.;52:143-152 2019).

A1AT comprises a flexible, solvent-accessible region known as the reactive center loop (RCL). Via this region, the protein interacts with its target, elastase. The sulfur atom of the amino acid methionine (M) easily reacts with oxygen species. For A1AT, it has been shown that the M residues 351 and 358 (situated in the RCL region) are readily oxidized, and that this event renders the protein inactive. By replacing these specific residues with non-oxidizable amino acids (such as leucine (L), isoleucine (I) or valine (V)), the protein became resistant against oxidation (Taggart et al., J. Biol. Chem.;275:27258-27265 2000, Griffiths et al., Biochemistry;41:6245-6252 2002, Pirooznia et al., Theor. Biol. Med. Model.;10:36 2013, Silberstein et al., Free Radical Bio. Med.;120:303-310 2018, Zhu et al., FEBS Open Bio; 8:1711-1721 2018).

However, several proteases, including a collection of matrix metalloproteinases (MMPs), have been reported to cleave A1AT in the RCL region, thereby inactivating the protein. Some of these are reported to cleave A1AT between residue 352 (phenylalanine, F) and residue 353 (L) (Sires et al., Biochem. Biophys. Res. Commun.;204:613-620 1994, Zhang et al., Biochim. Biophys. Acta;1199:224-228 1994, Nelson et al., Anal. Biochem.;260:230-236 1998, Taggart et al., J. Biol. Chem.;276:33345-33352 2001, Nie et al., Exp. Cell Res.;296:145-150 2004, Desrochers and Weiss, J. Clin. Invest.;81:1646-1650 1988).

Thus, there is a need to provide improved, protease-resistant variants of A1AT for use in treatment of A1AT deficiency and other diseases and disorders.

### SUMMARY OF THE INVENTION

It has been found by the present inventors that substitutions of L353 in A1AT provides protection against certain proteases, and combined with substitutions of M351 and M358, which are known to provide resistance to oxidation, the present inventors have identified A1AT variants which are resistant to both oxidation and proteolysis while retaining their elastase-inhibiting activity.

By producing the new variants in one of their previously developed cell lines, which decorate the protein with human N-glycans, the inventors have enabled the recombinant production of human-like oxidation-and proteolysis-resistant A1AT, suitable as a treatment for patients suffering from A1AT deficiency.

So, in a first aspect, the present invention relates to a variant of alpha-1-antitrypsin, wherein
(a) the variant has at least 90% sequence identity to SEQ ID NO:1,
(b) the amino acid at the position in SEQ ID NO: 1 corresponding to L353 is A, G, S or T, and
(c) the amino acids at the positions in SEQ ID NO: 1 corresponding to M351 and M358 are not M.

In some embodiments, in comparison to SEQ ID NO: 1, the variant has:
(a) an increased resistance to degradation by at least one protease,
(b) a retained or improved anti-elastase activity,
(c) an increased resistance to oxidation by hydrogen peroxide, and/or (
d) a combination of (a) and (b), (a) and (c), (b) and (c) and all of (a) to (c).

In some further embodiments, the at least one protease is selected from matrix metalloproteinases (MMP)-7,-8, and-9.

In some embodiments, the amino acid at the position in SEQ ID NO: 1 corresponding to L353 is A, S or T; such as A.

In some embodiments, the amino acids at the positions in SEQ ID NO: 1 corresponding to M351 and M358 are independently selected from V, I and L; such as V and I, such as V.

In some embodiments, the variant has the amino acid sequence of SEQ ID NO:1, except for a combination of amino acid substitutions selected from;
(i) L353A, M351V and M358V,
(ii) L353G, M351V and M358V,
(iii) L353S, M351V and M358V,
(iv) L353T, M351V and M358V,
(v) L353A, M351I and M358I,
(vi) L353G, M351I and M358I,
(vii) L353S, M351I and M358I,
(viii) L353T, M351I and M358I,
(ix) L353A, M351L and M358L,
(x) L353G, M351L and M358L,
(xi) L353S, M351L and M358L,
(xii) L353T, M351L and M358L,
(xiii) L353A, M351V and M358I,
(xiv) L353G, M351V and M358I,
(xv) L353S, M351V and M358I,
(xvi) L353T, M351V and M358I,
(xvii) L353A, M351I and M358V,
(xviii) L353G, M351I and M358V,
(xix) L353S, M351I and M358V,
(xx) L353T, M351I and M358V,
(xxi) L353A, M351L and M358V,
(xxii) L353G, M351L and M358V,
(xxiii) L353S, M351L and M358V,
(xxiv) L353T, M351L and M358V,
(xxv) L353A, M351V and M358L,
(xxvi) L353G, M351V and M358L,
(xxvii) L353S, M351V and M358L,
(xxviii) L353T, M351V and M358L,
(xxix) L353A, M351L and M358I,
(xxx) L353G, M351L and M358I,
(xxxi) L353S, M351L and M358I,
(xxxii) L353T, M351L and M358I,
(xxxiii) L353A, M351I and M358L,
(xxxiv) L353G, M351I and M358L,
(xxxv) L353S, M351I and M358L, and
(xxxvi) L353T, M351I and M358L.

In some embodiments, the variant is glycosylated and has a glycan profile with predominantly bi-antennary 2,6-sialylated, non-fucosylated type glycans.

In a second aspect, the present invention relates to a polynucleotide comprising a nucleic acid encoding the variant according to the first aspect.

In a third aspect, the present invention relates to a vector comprising the polynucleotide according to the second aspect.

In a fourth aspect, the present invention relates to a host cell comprising the vector according to the third aspect.

In some embodiments, the host cell is selected from the group consisting of a Chinese Hamster Ovary (CHO) cell, such as a CHO-K1, CHO-S or DG44 cell; a Baby Hamster Kidney (BHK) cell; a COS cell; a HEK293 cell; an NS0 cell; an SP2/0 cell; an YB2/0 cell; a HUVEC; a HKB cell; a PER-C6 cell; an NS0 cell; or a progeny or derivative of any of these cells.

In some embodiments, the host cell is a CHO cell wherein at least one of the endogenous genes Mgat4A, Mgat4B, Mgat5, St3Gal4, St3Gal6, SPPL3, and FUT8 are inactivated and/or downregulated.

In a fifth aspect, the present invention relates to a method for producing the variant according to the first aspect, the method comprising the steps of:
i) introducing the vector according to the third aspect into a suitable host cell,
ii) expressing the variant in the host cell, and
iii) isolating the variant.

In a sixth aspect, the variant, the polynucleotide or the vector according to any one of the first to third aspects or the fifth aspect is for use as a medicament.

In a seventh aspect, the variant, the polynucleotide or the vector according to any one of the first to third aspects or the fifth aspect is for use in the treatment of alpha-1-antitrypsin deficiency, cystic fibrosis or chronic obstructive pulmonary disease (COPD) in a subject in need thereof.

### LEGENDS TO THE FIGURES

FIG. 1: Glycoprofiles of A) plasma-purified human A1AT and B) wildtype A1AT expressed in glycoengineered CHO cells. The schematic of the most abundant type of glycan in both cases depicts the bi-antennary 2,6-sialylated, non-fucosylated (A2G2S2) glycan.

FIG. 2: Anti-elastase activity of A1AT L353 mutants. Supernatants of CHO cells transfected with plasmids encoding A1AT mutated at position 353 as indicated were tested for inhibition of elastase. As reference, a mixture of elastase and substrate without A1AT was included. The titer of A1AT in each CHO supernatant was determined (left). The inhibition assay was conducted using a four-fold dilution of the respective CHO supernatants (right).

FIG. 3: Evaluation of MMP-7-and MMP-9-mediated proteolytic degradation of three A1AT variants mutated at position 353. Plots of individual lanes of an SDS-gel in the size area of interest (between 55 and 70 kDa) are displayed. Processing of A1AT by either of the two MMPs would lead to a decrease of approximately 4 kDa. The profiles of untreated A1AT variants (upper row) are shown for comparison. The peak at the left-hand side of each plot corresponds to unprocessed A1AT (larger molecular weight (MW)), and the peak at the right-hand side of some of the plots corresponds to cleaved A1AT (lower MW).

FIG. 4: Evaluation of MMP-7-mediated proteolytic degradation of three additional A1AT variants mutated at position 353. Plots of individual lanes of an SDS-gel in the size area of interest (between 55 and 70 kDa) are displayed. The profiles of untreated A1AT (upper row) are shown for comparison. The peak at the left-hand side of the plots corresponds to unprocessed A1AT (larger molecular weight (MW)), and the peak at the right-hand side of the plots corresponds to cleaved A1AT (lower MW).

FIG. 5: Partial coding and peptide sequences of A1AT variants (A-C). See Example 1 for details.

FIG. 6: Anti-elastase activity of additional oxidation-and proteolysis-resistant A1AT mutants. Supernatants of CHO cells transfected with plasmids encoding A1AT mutated at position 351, 353 and 358 as indicated (Table 7) were tested for inhibition of elastase. As reference, a mixture of elastase and substrate without A1AT was included. The inhibition assay was conducted in presence of either 125 nM or 250 nM A1AT variant.

### DETAILED DISCLOSURE OF THE INVENTION

### Definitions

“Alpha-1-antitrypsin” or “A1AT” refers to the mature version of the protein identified as UniProtKB-P01009 (A1AT_HUMAN; isoform 1, v3) and any potential variants thereof, such as natural variants and polymorphs thereof as described in P01009 (accessed on 30 Sep. 2021). It is a protease inhibitor belonging to the serine protease inhibitor (serpin) superfamily. The protein has “anti-elastase activity”, meaning that it inactivates neutrophil elastase by binding to it. The mature version lacks the signal peptide corresponding to amino acids 1-24 of P01009-1 and is set forth herein as SEQ ID NO: 1, to which all amino acid positions refer.

A “variant” of a parent or reference protein comprises one or more mutations, such as amino acid substitutions, insertions and deletions, as compared to the parent or reference protein. Typically, the variant has a high sequence identity to the amino acid sequence of the parent or reference protein (e.g., at least about 70%, such as at least about 80%, such as at least about 90%, such as at least about 95%, such as at least about 99%, over at least the catalytically active portion, optionally over the full length).

Unless otherwise stated, the term “sequence identity” for nucleotide and amino acid sequences as used herein refers to the sequence identity calculated as (n_{ref}−n_{dif})·100/n_{ref}, wherein n_{dif} is the total number of non-identical nucleotides or amino acid residues in the two sequences when aligned and wherein n_{ref} is the number of nucleotides or amino acid residues in one of the sequences. Hence, the nucleotide sequence AGGTCCTA will have a sequence identity of 87.5% with the sequence AGTTCCTA (n_{dif}=1 and n_{ref}=8). The sequence identity can be determined by conventional methods, e.g., Smith and Waterman, (1981), Adv. Appl. Math. 2:482, by the ‘search for similarity’ method of Pearson & Lipman, (1988), Proc. Natl. Acad. Sci. USA 85:2444, using the CLUSTAL W algorithm of Thompson et al., (1994), Nucleic Acids Res 22:467380, by computerized implementations of these algorithms (GAP, BESTFIT, FASTA, and TFASTA in the Wisconsin Genetics Software Package, Genetics Computer Group). The BLAST algorithm (Altschul et al., (1990), Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

A residue in one amino acid sequence which “corresponds to” a specific reference residue in a reference amino acid sequence is the residue which aligns with the reference residue.

A “protease” is an enzyme which catalyses proteolysis—i.e., the breakdown or “degradation” of proteins into smaller polypeptides or single amino acids. This is done by cleaving the peptide bonds of the proteins by hydrolysis. Examples of proteases include matrix metalloproteinases, trypsin, chymotrypsin, and carboxypeptidases.

“Matrix metalloproteinases” or “MMPs” are calcium-dependent zinc-containing endopeptidases which degrade inter alia various extracellular matrix proteins. Examples of MMPs include MMP-7, -8, and -9.

As used herein, “vector” refers to any genetic element capable of serving as a vehicle of genetic transfer, expression, or replication for an exogenous nucleic acid sequence in a host cell. For example, a vector may be an artificial chromosome or a plasmid and may be capable of stable integration into a host cell genome, or it may exist as an independent genetic element (e.g., episome, plasmid). A vector may exist as a single nucleic acid sequence or as two or more separate nucleic acid sequences. Vectors may be single copy vectors or multicopy vectors when present in a host cell.

The term “host cell” refers to any cell into which an exogenous nucleic acid sequence can be introduced and expressed, typically via an expression vector. The host cell may, for example, be a wild-type cell isolated from its natural environment, a mutant cell identified by screening, a cell of a commercially available strain, or a genetically engineered cell or mutant cell, comprising one or more other exogenous and/or heterologous nucleic acid sequences than those of the invention. As used herein, the term “exogenous” means that the referenced item is not normally present in the host cell in question. The term “endogenous” means that the referenced item is normally present in or native to the host cell in question.

The term “MGAT4A” as used herein refers to the gene encoding Mannosyl (Alpha-1,3-)-Glycoprotein Beta-1,4-N-Acetylglucosaminyltransferase, Isozyme A (see Expasy enzyme entry: EC 2.4.1.145); the term “MGAT4B” as used herein refers to the gene encoding Mannosyl (Alpha-1,3-)-Glycoprotein Beta-1,4-N-Acetylglucosaminyltransferase, Isozyme B (EC 2.4.1.145); the term “MGAT5” as used herein refers to the gene encoding Mannosyl (Alpha-1,6-)-Glycoprotein Beta-1,6-N-Acetyl-Glucosaminyltransferase (EC 2.4.1.155); the term “ST3GAL4” as used herein refers to the gene encoding ST3 Beta-Galactoside Alpha-2,3-Sialyltransferase 4 (EC 2.4.99.9); the term “ST3GAL6” as used herein refers to the gene encoding ST3 Beta-Galactoside Alpha-2,3-Sialyltransferase 6 (EC 2.4.99.9); the term “SPPL3” as used herein refers to the gene encoding Signal Peptide Peptidase Like 3 (EC 3.4.23.-); the term “FUT8” as used herein refers to the gene encoding Fucosyltransferase 8 (EC 2.4.1.68 4). For further details, see WO 2019/105770 A1, which is hereby incorporated by reference in its entirety.

The term “inactivated and/or downregulated” refers to a modification of a mammalian host cell, wherein some specific genes are either knocked out, downregulated, or completely or partially inactivated in any other way, such as by miRNA post translational silencing. Preferably this inactivation is a complete inactivation with no measurable sign of expression of the inactivated gene. Suitable techniques to silence/knockout are very well described in the art and known to the person skilled in the art, e.g., as described in WO2015092737.

The term “introduction”, as used herein in the context of introducing a gene into a host cell, refers to any method of adding a transgene to a host cell, including methods such as transfection, transformation and transduction.

The term “expression”, as used herein, refers to the process in which a gene is transcribed into mRNA, and may optionally include the subsequent translation of the mRNA into an amino acid sequence, i.e., a protein or polypeptide.

An “isolated” molecule is a molecule that is the predominant species in the composition wherein it is found with respect to the class of molecules to which it belongs (i.e., it makes up at least about 5% of the type of molecule in the composition and typically will make up at least about 10%, at least about 20%, at least about 30%, at least about 40%, at least about 50%, at least about 60%, at least about 70%, at least about 80%, at least about 85%, at least about 90%, at least about 95%, or more of the species of molecules, e.g., peptides, in the composition). Commonly, a composition of a peptide molecule will exhibit 98%-99% homogeneity for peptide molecules in the context of all present peptide species in the composition or at least with respect to substantially active peptide species in the context of proposed use.

In the context of the present invention, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

The terms “patient” and “subject” refer to any human that may be treated using the methods of the present invention.

The diseases “alpha-1-antitrypsin deficiency”, “cystic fibrosis”, and “chronic obstructive pulmonary disease” or “COPD” are defined according to the relevant diagnostic criteria set out by the relevant clinical authorities.

### Specific Embodiments of the Invention

As described herein, the present inventors have discovered a way of producing improved variants of A1AT, particularly variants which can be more suitable for treating patients suffering from A1AT deficiency. For example, it is possible to make the protein protease-resistant by substituting the L in the position corresponding to position 353 with any one of alanine (A), glycine (G), serine(S), and threonine (T), while the protein retains its ability to inhibit elastase, even though the sites at which proteases cleave and inactivate A1AT are adjacent to the residues that interact with and thereby inhibit elastase. This discovery provides for A1AT variants which can be more resistant to cleavage by MMPs and/or other proteases, but still inhibit elastase. This may, in turn, contribute to prolonging the half-life of functional A1AT in the body.

The present inventors have generated A1AT variants comprising substitutions of L353 in combination with substitutions of M351 and M358 and have now shown that variants comprising substitutions in both L353 and M351 and M358 are both oxidation- and protease-resistant while retaining their anti-elastase activity. Furthermore, the new variants can be expressed in glycoengineered CHO cells, which add “humanized” N-glycans to the A1AT variants, in the sense that the N-glycans on the protein are similar or identical to those present on natural A1AT in human plasma, thus decreasing the risk of anti-drug immune responses. Moreover, recombinantly produced A1AT is more cost-effective to produce in large quantities and allows for a more homogenous and reproducible product. Also, since it is not dependent on donor blood, recombinant A1AT avoids the risk of transmitting blood-borne diseases.

Thus, the present invention provides A1AT variants suitable for use as medicaments, e.g., in the treatment of A1AT deficiency, cystic fibrosis and COPD.

In one aspect, the present invention relates to a variant of A1AT which carries amino acid substitutions in the positions corresponding to positions L353, M351 and M358 in SEQ ID NO:1.

Human A1AT (SEQ ID NO:1) is a protein of 394 amino acids:
EDPQGDAAQKTDTSHHDQDHPTFNKITPNLAEFAFSLYRQLAHQSNSTNIFFSPVSIATAFAMLSLGTKA DTHDEILEGLNFNLTEIPEAQIHEGFQELLRTLNQPDSQLQLTTGNGLFLSEGLKLVDKFLEDVKKLYHSEA FTVNFGDTEEAKKQINDYVEKGTQGKIVDLVKELDRDTVFALVNYIFFKGKWERPFEVKDTEEEDFHVDQ VTTVKVPMMKRLGMFNIQHCKKLSSWVLLMKYLGNATAIFFLPDEGKLQHLENELTHDIITKFLENEDRRS ASLHLPKLSITGTYDLKSVLGQLGITKVFSNGADLSGVTEEAPLKLSKAVHKAVLTIDEKGTEAAGAMFLEA IPMSIPPEVKFNKPFVFLMIEQNTKSPLFMGKVVNPTQK

In some embodiments, the A1AT variant has at least 90% sequence identity to SEQ ID NO: 1, such as at least 91% sequence identity to SEQ ID NO: 1, such as at least 92%, such as at least 93%, such as at least 94%, such as at least 95%, such as at least 96%, such as at least 97%, such as at least 98%, or such as at least 99% sequence identity to SEQ ID NO: 1.

In some embodiments, the amino acid at the position in SEQ ID NO: 1 corresponding to L353 is A. In some embodiments, the amino acid at the position in SEQ ID NO: 1 corresponding to L353 is G. In some embodiments, the amino acid at the position in SEQ ID NO: 1 corresponding to L353 is S. In some embodiments, the amino acid at the position in SEQ ID NO: 1 corresponding to L353 is T.

In some embodiments, the amino acids at the positions in SEQ ID NO: 1 corresponding to M351 and M358 are identical. In some embodiments, the amino acids at the positions in SEQ ID NO: 1 corresponding to M351 and M358 are different from each other.

In some embodiments, the amino acid at the position in SEQ ID NO: 1 corresponding to M351 is A. In some embodiments, the amino acid at the position in SEQ ID NO: 1 corresponding to M351 is R. In some embodiments, the amino acid at the position in SEQ ID NO: 1 corresponding to M351 is N. In some embodiments, the amino acid at the position in SEQ ID NO:1 corresponding to M351 is D. In some embodiments, the amino acid at the position in SEQ ID NO: 1 corresponding to M351 is C. In some embodiments, the amino acid at the position in SEQ ID NO: 1 corresponding to M351 is Q. In some embodiments, the amino acid at the position in

SEQ ID NO: 1 corresponding to M351 is E. In some embodiments, the amino acid at the position in SEQ ID NO:1 corresponding to M351 is G. In some embodiments, the amino acid at the position in SEQ ID NO: 1 corresponding to M351 is H. In some embodiments, the amino acid at the position in SEQ ID NO: 1 corresponding to M351 is I. In some embodiments, the amino acid at the position in SEQ ID NO: 1 corresponding to M351 is L. In some embodiments, the amino acid at the position in SEQ ID NO: 1 corresponding to M351 is K. In some embodiments, the amino acid at the position in SEQ ID NO: 1 corresponding to M351 is F. In some embodiments, the amino acid at the position in SEQ ID NO:1 corresponding to M351 is P. In some embodiments, the amino acid at the position in SEQ ID NO: 1 corresponding to M351 is S. In some embodiments, the amino acid at the position in SEQ ID NO: 1 corresponding to M351 is T. In some embodiments, the amino acid at the position in SEQ ID NO: 1 corresponding to M351 is W. In some embodiments, the amino acid at the position in SEQ ID NO: 1 corresponding to M351 is Y. In some embodiments, the amino acid at the position in SEQ ID NO: 1 corresponding to M351 is V.

In some embodiments, the amino acid at the position in SEQ ID NO: 1 corresponding to M358 is A. In some embodiments, the amino acid at the position in SEQ ID NO: 1 corresponding to M358 is R. In some embodiments, the amino acid at the position in SEQ ID NO: 1 corresponding to M358 is N. In some embodiments, the amino acid at the position in SEQ ID NO: 1 corresponding to M358 is D. In some embodiments, the amino acid at the position in SEQ ID NO: 1 corresponding to M358 is C. In some embodiments, the amino acid at the position in SEQ ID NO: 1 corresponding to M358 is Q. In some embodiments, the amino acid at the position in SEQ ID NO: 1 corresponding to M358 is E. In some embodiments, the amino acid at the position in SEQ ID NO: 1 corresponding to M358 is G. In some embodiments, the amino acid at the position in SEQ ID NO: 1 corresponding to M358 is H. In some embodiments, the amino acid at the position in SEQ ID NO: 1 corresponding to M358 is I. In some embodiments, the amino acid at the position in SEQ ID NO: 1 corresponding to M358 is L. In some embodiments, the amino acid at the position in SEQ ID NO: 1 corresponding to M358 is K. In some embodiments, the amino acid at the position in SEQ ID NO: 1 corresponding to M358 is F. In some embodiments, the amino acid at the position in SEQ ID NO:1 corresponding to M358 is P. In some embodiments, the amino acid at the position in SEQ ID NO: 1 corresponding to M358 is S. In some embodiments, the amino acid at the position in SEQ ID NO: 1 corresponding to M358 is T. In some embodiments, the amino acid at the position in SEQ ID NO: 1 corresponding to M358 is W. In some embodiments, the amino acid at the position in SEQ ID NO: 1 corresponding to M358 is Y. In some embodiments, the amino acid at the position in SEQ ID NO: 1 corresponding to M358 is V.

In preferred embodiments, the amino acids at the positions in SEQ ID NO: 1 corresponding to M351 and M358 are independently selected from V, I, and L, such as from V and I. In preferred embodiments, the amino acid at the position in SEQ ID NO: 1 corresponding to M351 is selected from V, I, and L, such as from V and I. In preferred embodiments, the amino acid at the position in SEQ ID NO: 1 corresponding to M358 is selected from V, I, and L, such as from V and I.

In some preferred embodiments, the variant comprises a combination of amino acid substitutions selected from;
(i) L353A, M351V and M358V,
(ii) L353G, M351V and M358V,
(iii) L353S, M351V and M358V,
(iv) L353T, M351V and M358V,
(v) L353A, M351I and M358I,
(vi) L353G, M351I and M358I,
(vii) L353S, M351I and M358I,
(viii) L353T, M351I and M358I,
(ix) L353A, M351L and M358L,
(x) L353G, M351L and M358L,
(xi) L353S, M351L and M358L,
(xii) L353T, M351L and M358L,
(xiii) L353A, M351V and M358I,
(xiv) L353G, M351V and M358I,
(xv) L353S, M351V and M358I,
(xvi) L353T, M351V and M358I,
(xvii) L353A, M351I and M358V,
(xviii) L353G, M351I and M358V,
(xix) L353S, M351I and M358V,
(xx) L353T, M351I and M358V,
(xxi) L353A, M351L and M358V,
(xxii) L353G, M351L and M358V,
(xxiii) L353S, M351L and M358V,
(xxiv) L353T, M351L and M358V,
(xxv) L353A, M351V and M358L,
(xxvi) L353G, M351V and M358L,
(xxvii) L353S, M351V and M358L,
(xxviii) L353T, M351V and M358L,
(xxix) L353A, M351L and M358I,
(xxx) L353G, M351L and M358I,
(xxxi) L353S, M351L and M358I,
(xxxii) L353T, M351L and M358I,
(xxxiii) L353A, M351I and M358L,
(xxxiv) L353G, M351I and M358L,
(xxxv) L353S, M351I and M358L, and
(xxxvi) L353T, M351I and M358L.

In some embodiments, the amino acid at the position in SEQ ID NO: 1 corresponding to M358 is not L.

The variants according to any aspect or embodiment described herein may be produced in the form of fusion proteins, wherein the variant is fused to a different peptide, e.g., a tag for purification, such as a His-tag, optionally wherein the tag is cleaved off before the protein is to be administrated to a patient.

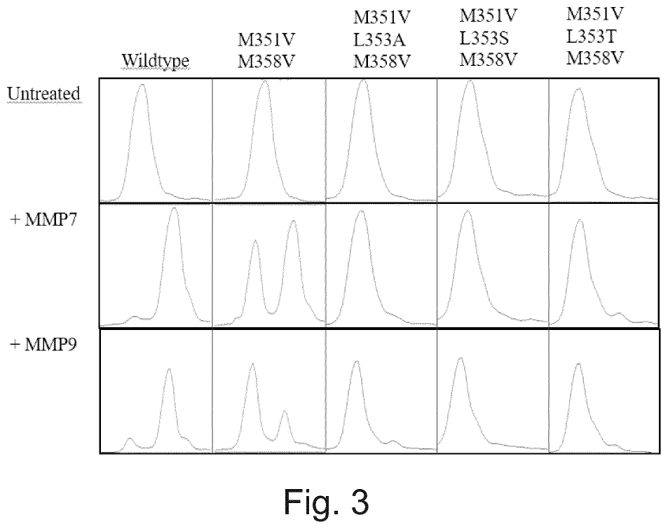

In some embodiments, the variant according to any aspect or embodiment described herein has an increased resistance to degradation by at least one protease. The protease may be an MMP, such as MMP-7, -8 or -9. For example, in some embodiments, the variant may be partly or fully protected against degradation by one or more proteases, as determined, e.g., by exposing the variant to one or more proteases and then running the resulting protein solution on an SDS-gel to determine if there is a shift in the size of the protein, indicating that cleavage has occurred (see e.g. FIG. 3). In this way the degree of cleavage may be calculated, e.g., by determining percentages of non-cleaved and cleaved protein from the peaks on the gel (see e.g. Table 5). The degree of cleavage of the variant calculated this way should then be compared to the degree of cleavage of a control protein, e.g. the A1AT WT protein of SEQ ID NO: 1. In some embodiments, a majority of the variant protein, such as at least 60%, such as at least 70%, such as at least 80%, such as at least 90%, such as at least 95%, and such as at least 99% of the protein remains non-cleaved after protease treatment.

In some embodiments, the variant according to any aspect or embodiment described herein has a retained or improved anti-elastase activity. In some embodiments, the variant has a retained anti-elastase activity. In other embodiments, the variant has an increased anti-elastase activity. For example, in some embodiments, anti-elastase activity may be determined by the anti-elastase assay described in Example 1. IC₅₀-values for the elastase-inhibiting activity of an A1AT variant can then be determined and compared with an IC₅₀-value for a reference protein, such as the A1AT WT protein of SEQ ID NO: 1. By dividing the value for the reference protein with the value for the A1AT variant, the fold change can be calculated. For example, in some embodiments, wherein the A1AT variant has an increased elastase-inhibiting activity, the fold change may be at least 1.2, such as at least 1.5. As used herein, “retained” anti-elastase activity means a fold change value ≥0.8 and ≤1.2, such as about 0.8, about 0.9, about 1.0, about 1.1, or about 1.2. As used herein, “improved” anti-elastase activity means any fold change value >1.0, such as >1.1, >1.2 or higher. A “retained or improved” anti-elastase activity can include any fold change value equal to or higher than 0.8, 0.9, or 1.0.

In some embodiments, the variant according to any aspect or embodiment described herein has an increased resistance to oxidation as compared to the wildtype protein. In specific embodiments, the variant has an increased resistance to oxidation by hydrogen peroxide. Since oxidation is known to inhibit the function of the protein, the resistance to oxidation after treatment with hydrogen peroxide may be measured by the anti-elastase assay referred to above. Likewise, the fold change may be calculated as described above and may, in some embodiments, be at least 1.2, such as at least 1.5, such as at least 2.

In some embodiments, the variant according to any aspect or embodiment described herein both has an increased resistance to degradation by at least one protease, a retained or improved anti-elastase activity, and an increased resistance to oxidation; or any combination of any of these three features. A determination of whether a variant has any combination of these three features may be performed as summarized above and described in Example 1. For example, the variant may have a resistance to degradation by MMP-7 resulting in at least 99.9% of the protein remaining non-cleaved, an increased resistance to oxidation by H₂O₂ resulting in a fold change in the IC₅₀-value of the anti-elastase activity after H₂O₂ treatment of at least 2.4, and an overall fold change in the IC₅₀-value of the anti-elastase activity of 1.5, e.g., when tested according to the assays described in Example 1.

In a second aspect, the present invention relates to a polynucleotide comprising a nucleic acid encoding the variant according to the present invention.

The polynucleotide may comprise a variant of the gene SERPINA1 (NCBI gene ID: 5265), which encodes human A1AT. The polynucleotide may further comprise other components, such as a suitable promoter, a gene sequence encoding a purification tag, a gene encoding a transcription factor, etc.

In a third aspect, the present invention relates to a vector comprising the polynucleotide according to the present invention.

Standard recombinant DNA and molecular cloning techniques useful for construction of appropriate expression vectors and other recombinant or genetic modification techniques for practising the invention, are well known in the art and are described by, e.g., Green and Sambrook, Molecular Cloning, A Laboratory Manual, Cold Spring Harbor Laboratory Press (Cold Spring Harbor, N.Y.) (2012); by Silhavy, T. J., Bennan, M. L. and Enquist, L. W. Experiments with Gene Fusions; Cold Spring Harbor Laboratory: Cold Spring Harbor, New York, 1984; by Ausubel et al., Short Protocols in Molecular Biology, Current Protocols, John Wiley and Sons (New Jersey) (2002), and references cited herein. Appropriate vectors are available commercially through, for example, the American Type Culture Collection (ATCC), Rockville, MD.

Preferred vectors for use in the present invention are expression vector molecules into which one or more genes encoding A1AT variant(s) can be inserted, in proper orientation and proximity to expression control elements resident in the expression vector molecule, so as to direct expression of one or more A1AT variants when the vector molecule resides in an appropriate host cell. Such an expression control element may for example be a promoter. When the host cell is a CHO cell, a promoter useful for controlling the expression of A1AT in the CHO cell may for example be a viral promoter like pCMV, pRSV or pSV40 or a human promoter like pEF1alpha or pUbC, or a composite artificial promoter like mCMV-hEF1-HTLV and SV40-hCMV-HTLV. Suitable promoters are readily available from many commercial vendors, or from repositories like Addgene.

In a fourth aspect, the present invention relates to a host cell comprising the vector according to the present invention.

In a fifth aspect, the present invention relates to a method for producing the variant according to the present invention.

According to the present invention, the host cell is not limited to any particular type of cell. However, the preferred host cell is a eukaryotic cell, more preferably a mammalian cell, such as a CHO cell. In even more preferred embodiments, the host cell is a CHO cell which has been genetically modified to produce proteins with a “human-like” N-glycosylation profile.

It is known from previous glyco-analyses of human A1AT from various sources that plasma-purified A1AT is glycosylated with a fully sialylated bi-antennary structure without core fucosylation. In contrast, CHO-produced A1AT contains many different structures with a partially and fully sialylated bi-antennary structure with core fucosylation as the most dominant species. It has previously been found that a shift of the glycosylation profile of recombinantly produced serum glycoproteins, including A1AT, towards the predominant bi-antennary form found in human plasma may be accomplished by knocking out, or in any other way downregulating, a selected set of glycosylating enzymes (WO19105770A1). The following targets have been described for modification of CHO cells for this purpose:
1) Inactivation and/or downregulation of a series of enzymes; Mgat4A, Mgat4B, and Mgat5, that facilitates a decrease in branching.
2) Inactivation and/or downregulation of a series of enzymes; St3Gal3, St3Gal4, and St3Gal6, that facilitates the removal of CHO-specific alpha-2,3-sialylation.
3) Inactivation and/or downregulation of the enzyme SPPL3, which facilitates an increase in the half-life of glycosyltransferases in the Golgi.
4) Inactivation and/or downregulation of the enzyme FUT8 which facilitates the removal of core fucosylation.
5) An optional inactivation and/or downregulation of the enzyme B3GNT2 which may remove elongated antennas, and
6) The insertion of a gene encoding Beta-galactoside alpha-2,6-sialyltransferase 1 (St6gal1), which gene directs a human-type branching of sialic acids.

With these modifications, it can be accomplished to shift the glycosylation profile to the predominant bi-antennary form found in human plasma.

In some embodiments, the host cell according to the present invention is a CHO cell wherein at least one of the endogenous genes Mgat4A, Mgat4B, Mgat5, St3Gal4, St3Gal6, SPPL3, and FUT8 are inactivated and/or downregulated.

In some embodiments, the host cell according to the present invention is selected from the group consisting of: a CHO cell, such as a CHO-K1, CHO-S or DG44 cell; a Baby Hamster Kidney (BHK) cell; a COS cell; a HEK293 cell; an NS0 cell; an SP2/0 cell; an YB2/0 cell; a HUVEC; a HKB cell; a PER-C6 cell; an NS0 cell; or a progeny or derivative of any of these cells.

In some embodiments, the host cell according to the present invention is a cell line producing the variants of A1AT with a primary N-glycan structure that is a fully sialylated bi-antennary structure without core fucosylation, such as with more than 80%, such as 82%, such as 84%, such as 86%, such as 88%, such as 90% of the glycoproteins of interest produced being in with a fully sialylated bi-antennary structure without core fucosylation.

In some embodiments, the host cell according to the present invention has a glycan structure according to the structure A2G2S2 with the following pictorial representation (Formula I):

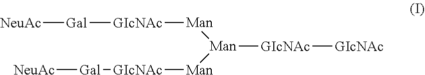

such as according to the structure (Formula II):

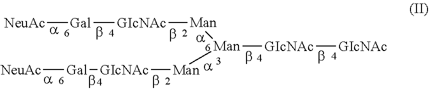

Also provided are pharmaceutical compositions comprising the A1AT variant according to any aspect or embodiment herein and a pharmaceutically acceptable carrier.

In some embodiments, the variant, the polynucleotide or the vector according to any aspect or embodiment herein may be for use in the treatment of alpha-1-antitrypsin deficiency, cystic fibrosis, COPD or any other disease, wherein elastase or other targets of A1AT play a role in the disease pathogenesis.

### EXAMPLE 1

### Materials and Methods

### Construction of Expression Vectors for Different A1AT Variants

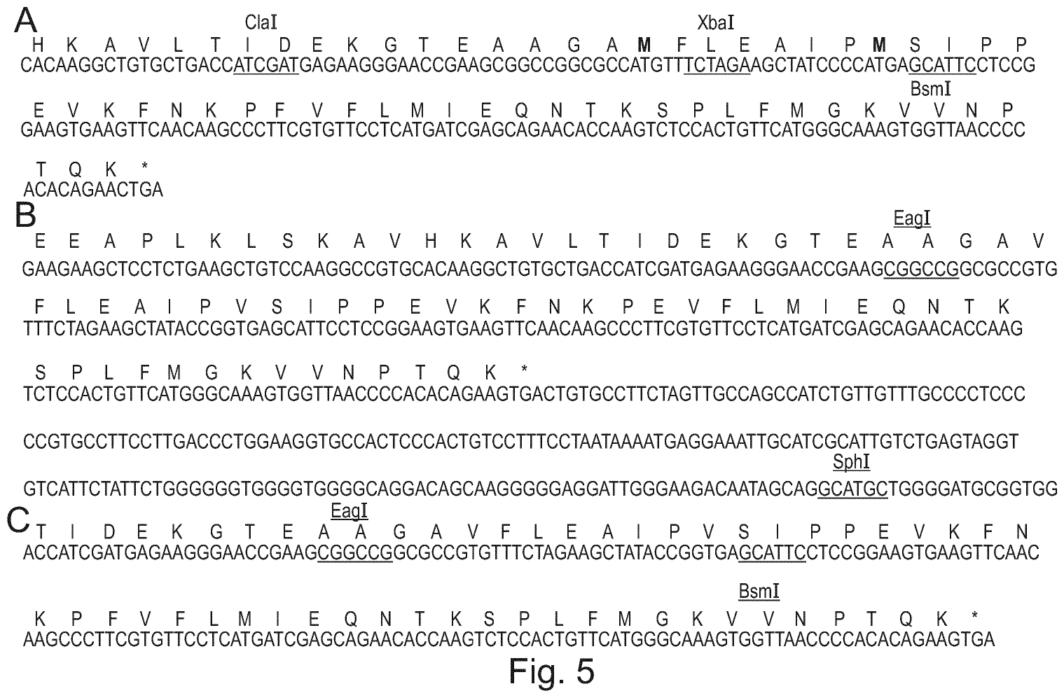

The coding sequence of A1AT (Uniprot P01009) was synthesized (Geneart/Thermo) with codons optimized for expression in hamster (Cricetulus griseus) cells, and with unique restriction sites flanking M351 (GAMF; ClaI and XbaI) and M358 (PMSI; XbaI and BsmI), see SEQ ID NO:2 (DNA) and SEQ ID NO:3 (peptide) in FIG. 5A, and cloned into a mammalian expression vector (A0019, plasmidID8671).

A0004, A0012 and A0016 were constructed from A0019 by sequentially first replacing the sequence between Xbal and BsmI restriction sites with annealed oligos that change M358 to V or I, and then replacing the sequence between ClaI and XbaI restriction sites with annealed oligos that change M351 to V or I (see Table 1 below).

TABLE 1

| SEQ | | | | Re- |
|---|---|---|---|---|
| ID | Oligo | Oligo | | striction |
| NO: | ID | name | Sequence | site |
| 4 | 23462 | M358V-top | ctagaagctataccggtgag | Xba1 |
| 5 | 23463 | M358V-bot | caccggtatagctt | Bsm1 |
| 6 | 23466 | M358I-top | ctagaagctatccccataag | Xba1 |
| 7 | 23467 | M358I-bot | tatggggatagctt | Bsm1 |
| 8 | 23508 | M351V-top2 | cgatgagaagggaaccgaagcggccggcgccgtgttt | Cla1 |
| 9 | 23509 | M351V-bot2 | ctagaaacacggcgccggccgcttcggttcccttctcat | Xba1 |
| 10 | 23512 | M351I-top2 | cgatgagaagggaaccgaagcggccggcgccatcttt | Cla1 |
| 11 | 23513 | M351I-bot2 | atgagaagggaaccgaagcggccggcgccatctttctag | Xba1 |

A0021, A0023 and A0024 were constructed from A0004 by replacing the C-terminal sequence of A1AT from the EagI restriction site just upstream of 351V and part of the downstream bGHpolyA sequence to an SphI restriction site with a PCR fragment generated from A0004 where 351V+353A/S/T is encoded in the forward primer (see SEQ ID NO:12 (DNA) and SEQ ID NO: 13 (peptide) in FIG. 5B and Table 2 below).

TABLE 2

| SEQ | | | | Re- |
|---|---|---|---|---|
| ID | | Primer | | striction |
| NO: | Primer ID | name | Sequence | site |
| 14 | PRIMER | serA1- | gttgttcggccggcgccgtgtttgccgaagctatac | EagI |
| | 12403 | M351V- | | |
| | | L353A | | |
| 15 | PRIMER | serA1- | gttgttcggccggcgccgtgttttccgaagctatac | EagI |
| | 12411 | M351V- | | |
| | | L353S | | |
| 16 | PRIMER | serA1- | gttgttcggccggcgccgtgtttaccgaagctatac | EagI |
| | 12412 | M351V- | | |
| | | L353T | | |
| 17 | PRIMER | bGH2-rv | caccgcatccccagcatgcctgctattgtcttc | SphI |
| | 12423 | | | |

A0025, A0028 and A0031 were constructed from A0004 by replacing the sequence between EagI and BsmI restriction sites with annealed oligos that introduce 353A flanked by 351M or V and 358M or V (see SEQ ID NO:18 (DNA) and SEQ ID NO:19 (peptide) in FIG. 5C and Table 3 below).

TABLE 3

| SEQ | | | | Re- |
|---|---|---|---|---|
| ID | | Primer | | striction |
| NO: | Primer ID | name | Sequence | site |
| 20 | PRIMER | A1AT- | ggccggcgccatgtttgcagaagctatccccatgag | EagI |
| | 14032 | MAM-top | | |
| 21 | PRIMER | A1AT- | catggggatagcttctgcaaacatggcgcc | BsmI |
| | 14033 | MAM-bot | | |
| 22 | PRIMER | A1AT- | ggccggcgccgtctttgcagaagctatccccatgag | EagI |
| | 14038 | VAM-top | | |
| 23 | PRIMER | A1AT-VAM- | catggggatagcttctgcaaagacggcgcc | BsmI |
| | 14039 | bot | | |
| 24 | PRIMER | A1AT- | ggccggcgccatgtttgcagaagctatccccgtcag | EagI |
| | 14044 | MAV-top | | |
| 25 | PRIMER | A1AT-MAV- | gacggggatagcttctgcaaacatggcgcc | BsmI |
| | 14045 | bot | | |

### Cell Culture

Glyco-engineered CHO-S cells were cultured according to the CHO-S guidelines provided by Life Technologies/Gibco and cells were thawed and cultured for 3 passages before chemical transfections using Fugene transfection reagent. Briefly, one day before transfection, cells were spun down at 300 g for 5 minutes and the complete CD CHO medium (containing 8 mM L-glutamine, 1% Antibiotic-Antimycotic agent and 0.2% Anti-clumping agent) was replaced with CD CHO medium without Anti-clumping agent (only containing 8 mM L-glutamine and 1% Antibiotic-Antimycotic agent). At the day of transfection, each transfection was accomplished in a 2L Corning shake flask with 500 mL CD-CHO medium (only containing 8 mM L-glutamine and 1% Antibiotic-Antimycotic agent) and a viable cell density at 800,000 cells/mL. The transfection complex was made by; first, in one tube, mixing 500 μg plasmid and Opti-PRO serum free medium to a final volume of 12.5 mL and secondly, in another tube, mixing 1.5 mL Fugene and 11 mL Opti-PRO serum free medium. The mixture from the first tube was added to the mixture from the second tube and the resulting plasmid/Fugene mixture was incubated for 5 minutes at room temperature. The plasmid/Fugene complex was then slowly added to the cell culture and the shake flask was placed in a CO₂-incubator (37° C., 5% CO₂ and 90% humidity) on a shaking platform with 120 rpm (25 mm throw). 72 hours post transfection, supernatants were harvested by centrifugation at 300g for 5 minutes and stored at −80° C.

### Purification

Supernatants were thawn in luke-warm water and centrifuged at 13,000g for 10 minutes at 4° C. The solutions were desalted by tangential flow filtration using a Centramate T-Series Cassette (Pall, DC010T02) mounted in a Centramate LV holder (Pall). First, the sample was up-concentrated to a final volume of 50 mL. Next, one diafiltration step was performed using 300 ml of 25 mM Bis-Tris buffer, pH 5.92. A1AT variants were purified from the desalted supernatants on an AKTA Pure protein purification system following a two-step anion exchange chromatography procedure. Both steps were performed using Q-sepharose columns, the first step with 25 mM Bis-Tris buffer (pH 5.92) and the second step with 10 mM Na2HPO4, 2 mM KH₂PO₄ (pH 8.0) as equilibration buffer. In the first step, the wash step contained 50 mM NaCl, while the protein was eluted with 150 mM NaCl. In the second step, the column was washed with 70 mM NaCl and the protein was eluted over a linear gradient from 70 to 200 mM NaCl. Fractions containing the protein of interest were concentrated on Amicon spin filter units (15 mL, 10 MWCO) and stored in aliquots at −80° C.

### A1AT Titer Determination

Expression levels of L353 mutants were determined using biolayer interferometry on an Octet RED96 instrument (Sartorius). All incubation steps in the Octet RED96 instrument were performed at 30° C. and a shaking speed of 1000 rpm. Streptavidin biosensors (Fortebio), hydrated in PBS, were coated with CaptureSelect™ biotin anti-A1AT conjugate (Thermo Scientific) at 5 μg/mL in PBS (600 s incubation), followed by a blocking step with 1 μg/ml biocytin in PBS (300 s incubation). Next, biosensors were equilibrated in spent CHO-S medium (120 s) and dipped in supernatants from glyco-engineered CHO cells expressing the various L353 mutants (300 s). A 2-fold serial dilution of A1AT (plasma-derived A1AT, Athens Research & Technology), prepared in spent CHO-S medium, was included as standard (highest concentration set at 100 μg/mL, 7 concentrations in total). Prior to analysis, all samples and standards were diluted 2-fold in PBS with additives present in a final concentration of 0.1% (w/v) BSA, 0.1% (v/v) Tween-20, and 500 mM NaCl. Biosensors were regenerated with 50 mM Tris, 2 M MgCl2, pH 7.5. Octet System Data Analysis 7.1 software was used to calculate binding rates and absolute A1AT concentrations.

Expression levels of the 32 additional mutants described in Table 7 were determined in a similar way. However, 1× kinetics buffer (Sartorius) was used for biosensor hydration, as well as during the loading, blocking and equilibration step. Moreover, samples were diluted 4-fold in 1× kinetics buffer instead of PBS to which additives were added in-house. Biosensors were regenerated with 10 mM glycine, pH 1.7.

### Anti-Elastase Assay

Anti-elastase activity was determined using the EnzChek™ Elastase Assay kit (Invitrogen™). A 1.5-fold dilution series of A1AT was prepared, typically with 1.6 μM of A1AT as the highest inhibitor concentration and with 10 concentrations in total (minimum of 120 μL per dilution). In a black, clear-bottom 96-well plate, first 100 μL of elastase working solution (0.5 U/mL) was added to the relevant number of wells (10+1 wells per A1AT variant, in duplicate). Next, 50 pL of each A1AT dilution was added to 10 consecutive wells containing the elastase working solution. As reference, 50 μL reaction buffer instead of A1AT solution was added to the 11th well. Using a multichannel pipette, 50 μL of substrate solution (0.1 mg/mL) was added to all 11 wells at the same time. Solutions were mixed by pipetting up and down. For background correction, the 12th well in a row was filled with 150 μL assay buffer and 50 μL substrate solution. The plate was incubated at room temperature protected from light for 50 min. Fluorescence intensity was measured in a fluorescence microplate reader (BioTek Synergy Mx), λₑₓ set at 485/9.0 nm and λₑₘ at 530/13.5 nm, 10 reads per well, gain at 80.

Data was processed by first subtracting the averaged background fluorescence from averaged intensities measured for each A1AT concentration and the reference. Next, the relative elastase activity detected in wells containing A1AT was normalized to the activity detected in the reference well. Relative elastase activity values were plotted against A1AT concentration in GraphPad Prism. IC₅₀-values were derived by non-linear regression using the “[Inhibitor] vs normalized response−variable slope” as model.

The inhibitory capacity of the 10 L353 mutants was determined using CHO supernatants instead of purified A1AT. The supernatants were up-concentrated such that all would contain approximately 50 μg/mL (1 μM) of AAT. The normalized samples (undiluted, 2-fold diluted or 4-fold diluted) were mixed with elastase and substrate, and relative elastase activities were determined.

The inhibitory capacity of the 32 additional triple mutants was also determined using CHO supernatants. Expression levels were around 60-80 μg/mL, hence, the supernatants were diluted to 50 μg/mL (1 μM) instead of up-concentrated. Relative elastase activities were determined upon mixing A1AT with elastase and substrate at a final A1AT concentration of either 250 nM or 125 nM.

### Glyco-Profiling

N-glycans were derivatized with GlycoWorks RapiFluor-MS N-Glycan Kit (Waters, Milford, MA) according to the manufacturer's instruction. Briefly, 12 μg purified protein was used for each sample. Labeled N-Glycans were analyzed by LC-MS as described previously (Grav et al., Biotechnology journal, 2015, Vol 10, p 1446-1456). Separation gradient from 30% to 43% 50 mM ammonium formate buffer and MS were run in positive mode. Amount of N-Glycan was measured by integrating the peaks with Thermo Xcalibur software (Thermo Fisher Scientific, Waltham, MA) giving the normalized, relative amount of the glycans.

### Assay Addressing the Susceptibility to Oxidation

A1AT variants were diluted to 2.5 mg/ml in oxidation buffer (50 mM K₂HPO₄, 100 mM KCl, 1 mM MgCl₂, PH 5.0). 2.5 μL of 200 mM H₂O₂ (prepared freshly by a 76-fold dilution of 50% w/v H₂O₂ solution in water) was added to 22.5 μL of 2.5 mg/mL A1AT. As negative control, 2.5 μL of MQ water was added instead of H₂O₂. Solutions were left at room temperature for 1 h. Next, the oxidation buffer was exchanged to PBS through two rounds of spin-filtration at 4° C. using Amicon spin filter units (0.5 mL, 10 MWCO). Following spin-filtration, the A1AT concentration of each sample was measured on a NanoDrop 2000 spectrophotometer (Protein A280, extinction coefficient of 0.44 (mg/mL)−1 cm−1) and adjusted to 1.6 μM. IC₅₀-values were determined as described above.

### Degradation of A1AT by MMP-7 and MMP-9

MMP-7 (ProSpec, cat #enz-271) and MMP-9 (ProSpec, cat #enz-438) were treated according to the manufacturer's instructions. A1AT variants (0.2 mg/mL) were incubated with 0.0125 U/mL MMP-7 or 40 μg/mL MMP-9, respectively, for 3 h at 37° C. Assay buffer: 50 mM Tris, 10 mM CaCl2, 0.15 M NaCl, 0.02% Brij-58, pH 7. Aliquots of each reaction mixture, containing 1.5 μg of A1AT, were loaded on a NuPAGE 4-12% Bis-Tris protein gel. Electrophoresis conditions: 35 min at 200 V in MES buffer. Gels were stained with InstantBlue Coomassie protein stain (Abcam), destained in water and scanned on an Amersham™ Imager 600. The relative amounts of non-cleaved vs. cleaved protein were determined using Amersham Imager 600 Analysis Software (v1.0.0) with the rolling ball method (radius of 5 mm) for background subtraction and automatic detection of bands at medium sensitivity.

### Results

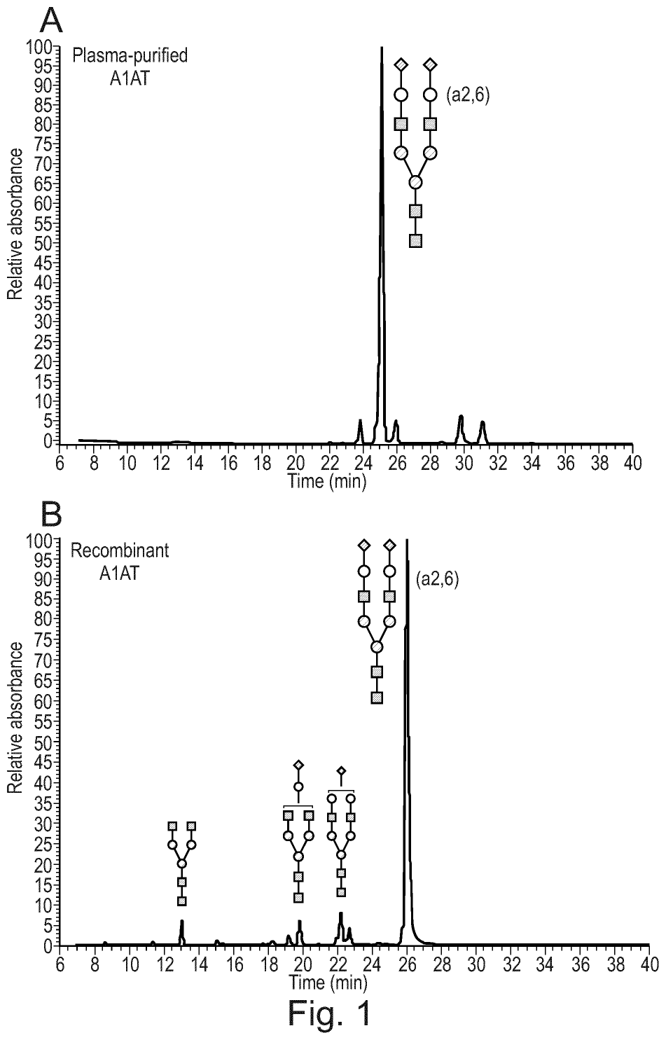

A1AT mutants in which residues M351 and M358 were substituted by V or I, were expressed in a glycoengineered CHO cell line, developed to provide glycoproteins with a homogenous, humanized N-glycan profile (Amann et al., Metab. Eng.; 52:143-152 2019). The expressed proteins were isolated from the cell supernatants according to a two-step anion-exchange purification procedure. A high level of purity was obtained as confirmed by SDS-PAGE gel electrophoresis followed by Coomassie staining. The identity of the N-glycans on the proteins was analyzed by mass spectrometric analysis and found to be in agreement with the expected profile (i.e. similar to the profile of plasma-purified A1AT; see comparison of the glycoprofiles of recombinantly expressed wildtype protein and plasma-purified A1AT in FIG. 1).

The effects of the amino acid substitutions on the mutants' ability to inhibit elastase were assessed by comparing the mutants' IC₅₀-values, obtained in the anti-elastase assay described above, with the one measured for the wildtype protein, expressed and purified under the exact same conditions (Table 4). The IC₅₀-values of the three mutants were all found to be slightly lower (i.e., the elastase-inhibiting activity was increased) than the IC₅₀-value of the wildtype protein. In addition, the anti-elastase activity of the mutants was unchanged after exposure to the oxidizing agent hydrogen peroxide, whereas the IC₅₀-value of the wildtype protein increased nearly two-fold.

TABLE 4
Anti-elastase activity of A1AT variants, before
and after treatment with oxidizing reagent H₂O₂.

| | | IC₅₀ before | IC₅₀ after |
|---|---|---|---|
| A1AT | | treatment | treatment |
| variant | Mutations | with H₂O₂ (nM)¹ | with H₂O₂ (nM)^{1, 2} |
| A0004 | M351V, M358V | 50.5 | 45.6 |
| | | (45.15 to 55.95) | (41.3 to 50.0) |
| A0012 | M351V, M358I | 50.8 | 47.78 |
| | | (45.4 to 56.2) | (41.4 to 54.2) |
| A0016 | M351I, M358V | 51.7 | 54.2 |
| | | (45.5 to 58.1) | (46.5 to 62.2) |
| Wildtype | — | 67.4 | 109.1 |
| | | (63.0 to 72.1) | (98.9 to 120.1) |
| Wildtype | — | 78.8 | 133.2 |
| | | (73.6 to 84.3) | (118.9 to 148.6) |

¹95% confidence interval is provided in parenthesis.
²Treatment with H₂O₂: A1AT (2.25 mg/mL) was incubated with 20 mM H₂O₂ for one hour at room temperature.

In order to identify mutants of A1AT resistant against the degradation by MMP-7, -8, and -9, the amino acid sequence of the reactive center loop (RCL) of A1AT was compared with the substrate specificity of these proteases (Eckhard et al., Matrix Biol.; 49:37-60 2016). It was noted that MMPs 7, 8, and 9 have a strong preference for an L residue at position P1′ of the cleavage site (according to the nomenclature of protease cleavage sites as formulated by Schechter and Berger (Schechter and Berger, Biochem. Biophys. Res. Com.;27:1567-162 1967)). This was in agreement with a previously reported observation that MMP-7, -8, and -9 cleave A1AT in the RCL region between residue F352 and residue L353 (Desrochers and Weiss, J. Clin. Invest.;81:1646-1650 1988). However, as this site is in close proximity to the reactive center of the protein (that is, the site where A1AT interacts with elastase), there was a significant risk that such a mutation would lead to loss of function of A1AT. In support of this, certain amino acid residues in the RCL of A1AT have previously been described to be critical for its anti-elastase activity (Scott and Sheffield, Protein Science;29:856-871 2020).

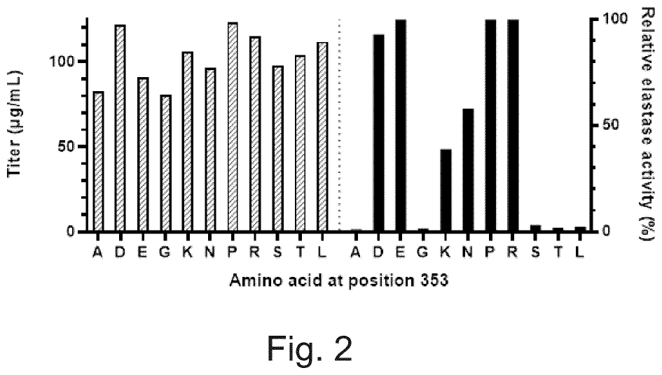

Ten mutants were generated, each mutant displaying a different amino acid at position 353: alanine (A), aspartic acid (D), glutamic acid (E), glycine (G), arginine (R), asparagine (N), proline (P), lysine (K), serine(S), and threonine (T). The L353 mutations were introduced in an A1AT variant which already contained the M351V and M358V mutations. The effects of the respective L353 mutations on A1AT's anti-elastase activity was assessed (FIG. 2). The mutants containing an E, P or R residue at position 353 had completely lost their ability to inhibit elastase. For three other mutants, those containing a D, K or N residue at position 353, the inhibitory activity towards elastase was significantly decreased. On the other hand, the mutants with an A, G, S or T residue at position 353 displayed similar anti-elastase activity as the A1AT variant containing the original L353 residue.

Three L353 mutants, shown to have improved or retained anti-elastase activity (A1AT-M351V/L353A/M358V, A1AT-M351V/L353S/M358V, and A1AT-M351V/L353T/M358V), were purified and treated with MMP-7 and MMP-9. For comparison, wildtype A1AT and the oxidation-resistant mutant A1AT-M351V/M358V were tested as well. Proteolytic cleavage was evaluated by gel electrophoresis. While wildtype A1AT got fully degraded by both MMP-7 and MMP-9, the L353 mutants remained unprocessed (FIG. 3 and Table 5). Surprisingly, also the oxidation-resistant mutant A1AT-M351V/M358V displayed less susceptibility towards proteolytic degradation than wildtype A1AT, though to a lesser extent than the L353 mutants.

The IC₅₀-values of the L353 mutants were determined in the anti-elastase assay (Table 6). Each mutant displayed an IC₅₀-value equal to or better than wildtype protein. A1AT-M351V/L353A/M358V was found to perform best, with a 1.2-1.5-fold lower IC₅₀-value compared to wildtype A1AT.

TABLE 5
Quantification of the degree of proteolytic
degradation of A1AT variants.

| | | MMP-7 | | MMP-9 | |
|---|---|---|---|---|---|
| | | non- | | non- | |
| | | cleaved | cleaved | cleaved | cleaved |
| | | (%) | (%) | (%) | (%) |
| A0019 | Wildtype | 4 | 96 | 11 | 89 |
| A0004 | M351V/M358V | 37 | 63 | 75 | 25 |
| A0021 | M351V/L353A/M358V | 100 | 0 | 94 | 6 |
| A0023 | M351V/L353S/M358V | 100 | 0 | 100 | 0 |
| A0024 | M351V/L353T/M358V | 95 | 5 | 100 | 0 |

TABLE 6
Anti-elastase activity of A1AT variants.

| A1AT variant | Mutations | IC₅₀ (nM)¹ | |
|---|---|---|---|
| A0021 | M351V, L353A, M358V | 74.6 | 78.2 |
| | | (70.0-79.2) | (75.5-81.0) |
| A0023 | M351V, L353S, M358V | 86.9 | 108.5 |
| | | (82.6-91.0) | (104.8-112.4) |
| A0024 | M351V, L353T, M358V | 98.9 | 115.1 |
| | | (95.2-103.0) | (112.2-118.1) |
| wildtype | — | 88.9 | 115.9 |
| | | (86.3-91.6) | (111.6-120.1) |

¹95% confidence interval provided in parenthesis. IC50 values were determined twice by the same operator on two separate days.

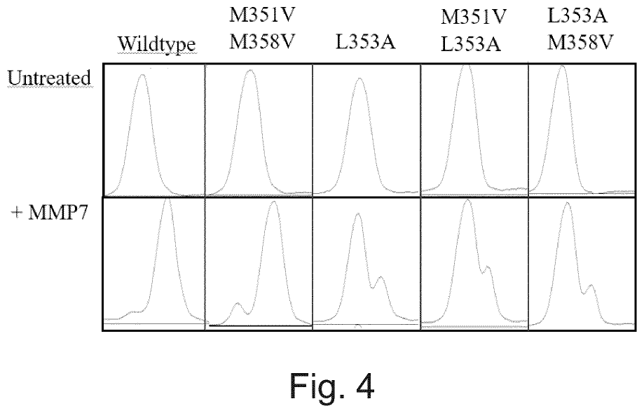

Three additional mutants were generated: one containing only the L353A mutation (A0025), one with the two mutations M351V and L353A (A0028), and one with the two mutations L353A and M358V (A0031). These mutants were also subjected to MMP-7 proteolysis (FIG. 4). A0025 showed as little degradation by MMP-7 as A0028 and A0031. Based on this result, it was concluded that mutating L353 alone was sufficient to render A1AT resistant against proteolytic degradation by MMP-7/-9.

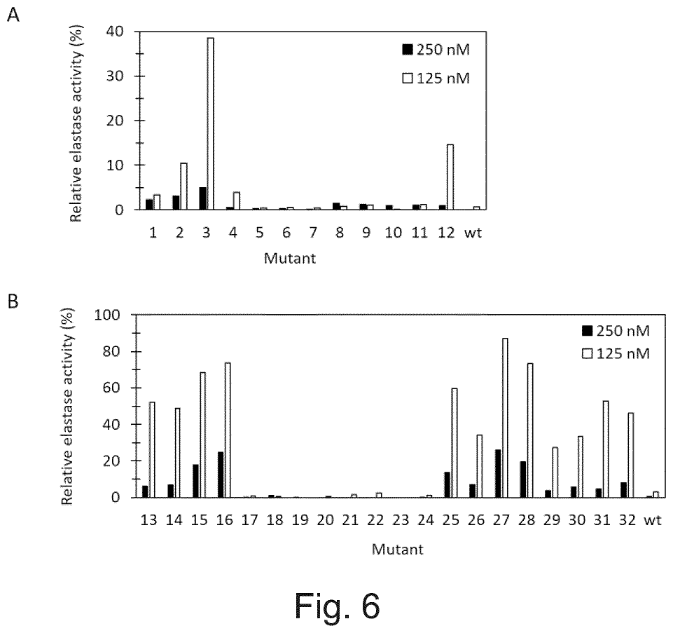

The L353A, L353G, L353S and L353T mutations were introduced in oxidation-resistant mutants A1AT-M351I/M358V, A1AT-M351V/M358I, A1AT-M351L/M358I, A1AT-M351L/M358L, A1AT-M351L/M3581, A1AT-M351L/M358V, A1AT-M351I/M358L, and A1AT-M351V/M358L (Table 7). All 32 triple mutants exhibited anti-elastase activity (FIG. 6). Mutants containing the M358L mutation were found to be less inhibiting, though, than wildtype A1AT or A1AT variants containing the M3581 or M358V mutation.

TABLE 7
IDs for 32 additional A1AT variants.

| ID | Mutations |
|---|---|
| 1 | A1AT-M351I, L353A, M358V |
| 2 | A1AT-M351I, L353G, M358V |
| 3 | A1AT-M351I, L353S, M358V |
| 4 | A1AT-M351I, L353T, M358V |
| 5 | A1AT-M351V, L353A, M358I |
| 6 | A1AT-M351V, L353G, M358I |
| 7 | A1AT-M351V, L353S, M358I |
| 8 | A1AT-M351V, L353T, M358I |
| 9 | A1AT-M351I, L353A, M358I |
| 10 | A1AT-M351I, L353G, M358I |
| 11 | A1AT-M351I, L353S, M358I |
| 12 | A1AT-M351I, L353T, M358I |
| 13 | A1AT-M351L, L353A, M358L |
| 14 | A1AT-M351L, L353G, M358L |
| 15 | A1AT-M351L, L353S, M358L |
| 16 | A1AT-M351L, L353T, M358L |
| 17 | A1AT-M351L, L353A, M358I |
| 18 | A1AT-M351L, L353G, M358I |
| 19 | A1AT-M351L, L353S, M358I |
| 20 | A1AT-M351L, L353T, M358I |
| 21 | A1AT-M351L, L353A, M358V |
| 22 | A1AT-M351L, L353G, M358V |
| 23 | A1AT-M351L, L353S, M358V |
| 24 | A1AT-M351L, L353T, M358V |
| 25 | A1AT-M351I, L353A, M358L |
| 26 | A1AT-M351I, L353G, M358L |
| 27 | A1AT-M351I, L353S, M358L |
| 28 | A1AT-M351I, L353T, M358L |
| 29 | A1AT-M351V, L353A, M358L |
| 30 | A1AT-M351V, L353G, M358L |
| 31 | A1AT-M351V, L353S, M358L |
| 32 | A1AT-M351V, L353T, M358L |

### LIST OF REFERENCES

Each reference listed below or cited elsewhere herein is hereby incorporated by reference in its entirety.
Amann, T. et al. (2019). Glyco-engineered CHO cell lines producing alpha-1-antitrypsin and C1 esterase inhibitor with fully humanized N-glycosylation profiles. Metab. Eng. 52, 143-152.
Banda et al. (1987). Interaction of mouse macrophage elastase with native and oxidized human alpha 1-proteinase inhibitor. J. Clin. Invest. 79, 1314-1317.
Borriello, F., and Krauter, K. S. (1991). Multiple murine alpha 1-protease inhibitor genes show unusual evolutionary divergence. PNAS 88, 9417-9421.
Desrochers, P. E., and Weiss, S. J. (1988). Proteolytic inactivation of alpha-1-proteinase inhibitor by a neutrophil metalloproteinase. J. Clin. Invest. 81, 1646-1650.
Griffiths, S. W., and Cooney, C. L. (2002). Relationship between protein structure and methionine oxidation in recombinant human α1-antitrypsin. Biochemistry 41, 6245-6252.
Nelson, D., Potempa, J., and Travis, J. (1998). Inactivation of α1-proteinase inhibitor as a broad screen for detecting proteolytic activities in unknown samples. Anal. Biochem. 260, 230-236.
Nie, J., and Pei, D. (2004). Rapid inactivation of alpha-1-proteinase inhibitor by neutrophil specific leukolysin/membrane-type matrix metalloproteinase 6. Exp. Cell Res. 296, 145-150.
Park et al. (2002). Peptide substrate specificities and protein cleavage sites of human endometase/matrilysin-2/matrix metalloproteinase-26. J. Biol. Chem. 277, 35168-75.
Pirooznia, N. et al. (2013). The design of a new truncated and engineered alpha1-antitrypsin based on theoretical studies: an antiprotease therapeutics for pulmonary diseases. Theor. Biol. Med. Model. 10:36.
Schulze, A. J. et al. (1991). Inhibitory activity and conformational transition of α1-proteinase inhibitor variants. Eur. J. Biochem. 202, 1147-1155.
Scott, B. M., and Sheffield, W. P. (2020). Engineering the serpin α1-antitrypsin: A diversity of goals and techniques. Protein Science. 29, 856-871.
Silberstein, D. Z. et al. (2018). An oxidation-resistant, recombinant alpha-1 antitrypsin produced in Nicotiana benthamiana. Free Radical Bio. Med. 120, 303-310.
Silva et al. (2016). Alpha-1-antitrypsin (SERPINA1) mutation spectrum: Three novel variants and haplotype characterization of rare deficiency alleles identified in Portugal. Respir. Med. 116, 8-18.
Sires, U. I. et al. (1994). Matrilysin is much more efficient than other matrix metalloproteinases in the proteolytic inactivation of α1-antitrypsin. Biochem Biophys Res. Commun. 204, 613-620.
Sosulski et al. (2020). Gene therapy for alpha 1-antitrypsin deficiency with an oxidant-resistant human alpha 1-antitrypsin. J. Clin. Invest. Insight 6, e135951.
Taggart, C. et al. (2000). Oxidation of either methionine 351 or methionine 358 in α1-antitrypsin causes loss of anti-neutrophil elastase activity. J. Biol. Chem. 275, 27258-27265.
Taggart, C. et al. (2001). Cathepsin B, L, and S cleave and inactivate secretory leucoprotease inhibitor. J. Biol. Chem. 276, 33345-33352.
Zhang, Z. et al. (1994). Proteolysis of human native and oxidized α1-proteinase inhibitor by matrilysin and stromelysin. Biochim. Biophys. Acta 1199, 224-228.
Zhu, W. et al. (2018). Oxidation-resistant and thermostable forms of alpha-1 antitrypsin from Escherichia coli inclusion bodies. FEBS Open Bio 8, 1711-1721.
CA1341165A1 (Chiron Corp.)
US4732973 A (Chiron Corp.)
US4752576 A (Chiron Corp.)
CA1341219 A1 (ZymoGenetics Inc.)
US6072029 A (Transgene SA)
WO8600337 A1 (Transgene SA)
WO9426781 A1 (Korea Green Cross Corp.)
WO2018183705 A1 (Cornell Univ.)
WO2019105770 A1 (Danmarks Tekniske Universitet)
WO2019148848 A1 (Univ Beijing)

The invention claimed is:

1. A variant of alpha-1-antitrypsin, wherein
(a) the variant has at least 90% sequence identity to SEQ ID NO:1,
(b) the amino acid at the position in SEQ ID NO:1 corresponding to L353 is A, G, S or T, and
(c) the amino acids at the positions in SEQ ID NO:1 corresponding to M351 and M358 are not M.

2. The variant according to claim 1, wherein, in comparison to SEQ ID NO:1, the variant has:
(a) an increased resistance to degradation by at least one protease,
(b) a retained or improved anti-elastase activity,
(c) an increased resistance to oxidation by hydrogen peroxide, and/or
(d) a combination of (a) and (b), (a) and (c), (b) and (c) and all of (a) to (c).

3. The variant according to claim 2, wherein the at least one protease is selected from matrix metalloproteinases (MMP)-7, -8, and -9.

4. The variant according to claim 1, wherein the amino acid at the position in SEQ ID NO:1 corresponding to L353 is A, S or T.

5. The variant according to claim 1, wherein the amino acids at the positions in SEQ ID NO:1 corresponding to M351 and M358 are independently selected from V, I and L.

6. The variant according to claim 1, wherein the variant comprises the amino acid sequence of SEQ ID NO:1, except for a combination of amino acid substitutions selected from:
(i) L353A, M351V and M358V,
(ii) L353G, M351V and M358V,
(iii) L353S, M351V and M358V,
(iv) L353T, M351V and M358V,
(v) L353A, M351I and M358I,
(vi) L353G, M351I and M358I,
(vii) L353S, M351I and M358I,
(viii) L353T, M351I and M358I,
(ix) L353A, M351L and M358L,
(x) L353G, M351L and M358L,
(xi) L353S, M351L and M358L,
(xii) L353T, M351L and M358L,
(xiii) L353A, M351V and M358I,
(xiv) L353G, M351V and M358I,
(xv) L353S, M351V and M358I,
(xvi) L353T, M351V and M358I,
(xvii) L353A, M351I and M358V,
(xviii) L353G, M351I and M358V,
(xix) L353S, M351I and M358V,
(xx) L353T, M351I and M358V,
(xxi) L353A, M351L and M358V,
(xxii) L353G, M351L and M358V,
(xxiii) L353S, M351L and M358V,
(xxiv) L353T, M351L and M358V,
(xxv) L353A, M351V and M358L,
(xxvi) L353G, M351V and M358L,
(xxvii) L353S, M351V and M358L,
(xxviii) L353T, M351V and M358L,
(xxix) L353A, M351L and M358I,
(xxx) L353G, M351L and M358I,
(xxxi) L353S, M351L and M358I,
(xxxii) L353T, M351L and M358I,
(xxxiii) L353A, M351I and M358L,
(xxxiv) L353G, M351I and M358L,
(xxxv) L353S, M351I and M358L, and
(xxxvi) L353T, M351I and M358L.

7. The variant according to claim 1, wherein the variant is glycosylated and has a glycan profile with predominantly bi-antennary 2,6-sialylated, non-fucosylated type glycans.

8. A polynucleotide comprising a nucleic acid encoding the variant according to claim 1.

9. A vector comprising the polynucleotide according to claim 8.

10. A host cell comprising the vector according to claim 9.

11. The host cell according to claim 10, wherein the host cell is selected from the group consisting of a Chinese Hamster Ovary (CHO) cell; a Baby Hamster Kidney (BHK) cell; a COS cell; a HEK293 cell; an NS0 cell; an SP2/0 cell; an YB2/0 cell; a HUVEC; a HKB cell; a PER-C6 cell; an NS0 cell; or a progeny or derivative of any of these cells.

12. The host cell according to claim 10, which is a CHO cell wherein at least one of the endogenous genes Mgat4A, Mgat4B, Mgat5, St3Gal4, St3Gal6, SPPL3, and FUT8 are inactivated and/or downregulated.

13. A method for producing the variant according to claim 1, the method comprising the steps of:
i) introducing the vector according to claim 9 into a suitable host cell,
ii) expressing the variant in the host cell, and
iii) isolating the variant.

14. (canceled)

15. A method of treating alpha-1-antitrypsin deficiency, cystic fibrosis or chronic obstructive pulmonary disease (COPD) comprising administering to a subject in need thereof the variant according to claim 1.

16. The variant according to claim 1, wherein the amino acid at the position in SEQ ID NO:1 corresponding to L353 is A.

17. The variant according to claim 1, wherein the amino acids at the positions in SEQ ID NO:1 corresponding to M351 and M358 are independently selected from V and I.

18. The variant according to claim 1, wherein the amino acids at the positions in SEQ ID NO:1 corresponding to M351 and M358 are V.

19. The host cell according to claim 10, wherein the host cell is a Chinese Hamster Ovary (CHO) cell selected from the group consisting of a CHO-K1 cell, a CHO-S cell, and a DG44 cell.


SEQUENCE LISTING (25 sequences)

SEQ ID NO: 1 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - DNA, 186 bp, synthetic construct
  misc_feature 1..186: note = Recombinant sequence
  source 1..186: mol_type = other DNA
cacaaggctgtgctgaccatcgatgagaagggaaccgaagcggccggcgccatgtttctagaagctatccccatgagcattcctccggaagtgaagttcaacaagcccttcgtgttcctcatgatcgagcagaacaccaagtctccactgttcatgggcaaagtggttaaccccacacagaagtga

SEQ ID NO: 3 - protein, 61 aa, synthetic construct
  REGION 1..61: note = Recombinant sequence
HKAVLTIDEKGTEAAGAMFLEAIPMSIPPEVKFNKPFVFLMIEQNTKSPLFMGKVVNPTQK

SEQ ID NO: 4 - DNA, 20 bp, synthetic construct
  misc_feature 1..20: note = Recombinant sequence
  source 1..20: mol_type = other DNA
ctagaagctataccggtgag

SEQ ID NO: 5 - DNA, 14 bp, synthetic construct
  misc_feature 1..14: note = Recombinant sequence
  source 1..14: mol_type = other DNA
caccggtatagctt

SEQ ID NO: 6 - DNA, 20 bp, synthetic construct
  misc_feature 1..20: note = Recombinant sequence
  source 1..20: mol_type = other DNA
ctagaagctatccccataag

SEQ ID NO: 7 - DNA, 14 bp, synthetic construct
  misc_feature 1..14: note = Recombinant sequence
  source 1..14: mol_type = other DNA
tatggggatagctt

SEQ ID NO: 8 - DNA, 37 bp, synthetic construct
  misc_feature 1..37: note = Recombinant sequence
  source 1..37: mol_type = other DNA
cgatgagaagggaaccgaagcggccggcgccgtgttt

SEQ ID NO: 9 - DNA, 39 bp, synthetic construct
  misc_feature 1..39: note = Recombinant sequence
  source 1..39: mol_type = other DNA
ctagaaacacggcgccggccgcttcggttcccttctcat

SEQ ID NO: 10 - DNA, 37 bp, synthetic construct
  misc_feature 1..37: note = Recombinant sequence
  source 1..37: mol_type = other DNA
cgatgagaagggaaccgaagcggccggcgccatcttt

SEQ ID NO: 11 - DNA, 39 bp, synthetic construct
  misc_feature 1..39: note = Recombinant sequence
  source 1..39: mol_type = other DNA
atgagaagggaaccgaagcggccggcgccatctttctag

SEQ ID NO: 12 - DNA, 435 bp, synthetic construct
  misc_feature 1..435: note = Recombinant sequence
  source 1..435: mol_type = other DNA
gaagaagctcctctgaagctgtccaaggccgtgcacaaggctgtgctgaccatcgatgagaagggaaccgaagcggccggcgccgtgtttctagaagctataccggtgagcattcctccggaagtgaagttcaacaagcccttcgtgttcctcatgatcgagcagaacaccaagtctccactgttcatgggcaaagtggttaaccccacacagaagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgg

SEQ ID NO: 13 - protein, 72 aa, synthetic construct
  REGION 1..72: note = Recombinant sequence
EEAPLKLSKAVHKAVLTIDEKGTEAAGAVFLEAIPVSIPPEVKFNKPFVFLMIEQNTKSPLFMGKVVNPTQK

SEQ ID NO: 14 - DNA, 36 bp, synthetic construct
  misc_feature 1..36: note = Recombinant sequence
  source 1..36: mol_type = other DNA
gttgttcggccggcgccgtgtttgccgaagctatac

SEQ ID NO: 15 - DNA, 36 bp, synthetic construct
  misc_feature 1..36: note = Recombinant sequence
  source 1..36: mol_type = other DNA
gttgttcggccggcgccgtgttttccgaagctatac

SEQ ID NO: 16 - DNA, 36 bp, synthetic construct
  misc_feature 1..36: note = Recombinant sequence
  source 1..36: mol_type = other DNA
gttgttcggccggcgccgtgtttaccgaagctatac

SEQ ID NO: 17 - DNA, 33 bp, synthetic construct
  misc_feature 1..33: note = Recombinant sequence
  source 1..33: mol_type = other DNA
caccgcatccccagcatgcctgctattgtcttc

SEQ ID NO: 18 - DNA, 171 bp, synthetic construct
  misc_feature 1..171: note = Recombinant sequence
  source 1..171: mol_type = other DNA
accatcgatgagaagggaaccgaagcggccggcgccgtgtttctagaagctataccggtgagcattcctccggaagtgaagttcaacaagcccttcgtgttcctcatgatcgagcagaacaccaagtctccactgttcatgggcaaagtggttaaccccacacagaagtga

SEQ ID NO: 19 - protein, 56 aa, synthetic construct
  REGION 1..56: note = Recombinant sequence
TIDEKGTEAAGAVFLEAIPVSIPPEVKFNKPFVFLMIEQNTKSPLFMGKVVNPTQK

SEQ ID NO: 20 - DNA, 36 bp, synthetic construct
  misc_feature 1..36: note = Recombinant sequence
  source 1..36: mol_type = other DNA
ggccggcgccatgtttgcagaagctatccccatgag

SEQ ID NO: 21 - DNA, 30 bp, synthetic construct
  misc_feature 1..30: note = Recombinant sequence
  source 1..30: mol_type = other DNA
catggggatagcttctgcaaacatggcgcc

SEQ ID NO: 22 - DNA, 36 bp, synthetic construct
  misc_feature 1..36: note = Recombinant sequence
  source 1..36: mol_type = other DNA
ggccggcgccgtctttgcagaagctatccccatgag

SEQ ID NO: 23 - DNA, 30 bp, synthetic construct
  misc_feature 1..30: note = Recombinant sequence
  source 1..30: mol_type = other DNA
catggggatagcttctgcaaagacggcgcc

SEQ ID NO: 24 - DNA, 36 bp, synthetic construct
  misc_feature 1..36: note = Recombinant sequence
  source 1..36: mol_type = other DNA
ggccggcgccatgtttgcagaagctatccccgtcag

SEQ ID NO: 25 - DNA, 30 bp, synthetic construct
  misc_feature 1..30: note = Recombinant sequence
  source 1..30: mol_type = other DNA
gacggggatagcttctgcaaacatggcgcc